(19) United States
(12) Patent Application Publication
(10) Pub. No.: US 2016/0077079 A1
(43) Pub. Date: Mar. 17, 2016

(54) THE CHILDREN'S HOSPITAL OF PHILADELPHIA

(71) Applicant: THE CHILDREN'S HOSPITAL OF PHILADELPHIA, Philadelphia, PA (US)

(72) Inventors: Struan F.A. Grant, Swarthmore, PA (US); Xia Qianghua, Drexel Hill, PA (US)

(21) Appl. No.: 14/775,358

(22) PCT Filed: Mar. 14, 2014
(86) PCT No.: PCT/US14/28803

§ 371 Date: Sep. 11, 2015

Related U.S. Application Data
(60) Provisional application No. 61/782,646, filed on Mar. 14, 2013.
(60) Provisional application No. 61/807,036, filed on Apr. 1, 2013.
(60) Provisional application No. 61/921,585, filed on Dec. 30, 2013.

(51) Int. Cl. G01N 33/50; A61K 45/06; A61K 31/166; A61K 31/7004; A61K 31/502; A61K 31/55

(52) U.S. Cl. CPC G01N 33/502; A61K 31/502; A61K 31/55; A61K 31/166; A61K 31/7004; A61K 45/06; G01N 2333/4706; G01N 2800/042

(57) ABSTRACT

Compositions and methods useful for the identification of therapeutic agents useful for the treatment of T2D are disclosed.

This application claims priority to U.S. Provisional Applications 61/782,646, 61/807,036 and 61/921,585 filed Mar. 14, 2013, Apr. 1, 2013 and Dec. 30, 2013, respectively. The entire disclosures of each of the aforementioned applications being incorporated herein by reference as though set forth in full.

### FIELD OF THE INVENTION

This invention relates to the fields of transcriptional regulation of metabolic disease and drug screening. More specifically, the invention provides the functional characterization of the transcriptional machinery bound across a TCF7L2 variant which plays a role in a variety of disorders, including without limitation, type 2 diabetes, cystic fibrosis related diabetes (CFRD), latent autoimmune diabetes in adults (LADA), gestational diabetes, islet antibody-negative diabetes in young patients, cardio- and micro-vascular disease and schizophrenia. Agents which disrupt formation of this complex should have efficacy in the treatment of such disorders.

### BACKGROUND OF THE INVENTION

Several publications and patent documents are cited throughout the specification in order to describe the state of the art to which this invention pertains. Each of these citations is incorporated herein by reference as though set forth in full.

Transcription factor 7-like 2 (TCF7L2) (T-cell specific, HMG-box) also known as HMG box transcription factor 4 or T-cell-specific transcription factor 4 is a member of the TCF/LEF family which is involved in the Wnt signaling pathway (van Es, Jay et al. 2005). The canonical Wnt pathway is initiated by Wnt ligands, a group of secreted glycoproteins that control stabilization of β-catenin. In the absence of ligand, cytoplasmic β-catenin binds to APC and Axin and is hyperphosphorylated by the kinases CKIα and GSK3β, ultimately resulting in ubiqitination and degradation by the proteosome. When Wnt proteins bind to a cell-surface receptor complex of a Frizzled family receptor and a coreceptor of the transmembrane LRP-5/6/arrow family, the destruction complex (CKIα, GSK3β, APC, Axin) fail to destroyl β-catenin, causing stabilization of β-catenin. The accumulated β-catenin is able to translocate into the nucleus and interact with TCF/LEF family transcription factors to activate the target gene expression (Logan and Nusse 2004). Wnt signaling pathways are evolutionarily conserved between C. elegans, Drosophila and vertebrates and play a variety of important roles in cell polarity, cell differentiation, embryonic development and many diseases such as cancer (Logan and Nusse 2004). TCF7L2 is the main effector of Wnt signaling (Ravindranath, O'Connell et al. 2008; Grove 2011). TCF7L2 can bind to the DNA motif in a sequence-specific manner and regulate Wnt target genes expression. It acts as a repressor or an activator. In the absence of β-catenin, TCF7L2 bind to Wnt-responsive elements to repress target gene transcription while β-catenin binding to TCF7L2 activates gene expression.

It is well known that the T-allele of the transcription factor 7-like 2 gene (TCF7L2) polymorphism rs7903146 is associated with type 2 diabetes (Grant, Thorleifsson et al. 2006; Saxena, Gianniny et al. 2006; Scott, Bonnycastle et al. 2006; Helgason, Palsson et al. 2007). Genotyping in African Americans supports the prevailing consensus that rs7903146 is a causal variant at this locus, conferring risk for type 2 diabetes (Palmer, Hester et al. 2011). However, the underlying mechanism of how the SNP rs7903146 regulates the function of TCF7L2 remains to be elucidated.

### SUMMARY OF THE INVENTION

In accordance with the present invention, an isolated binding complex comprising a SNP containing transcription factor 7-like 2 (TCF7L2) encoding nucleic acid, wherein said SNP is rs7903146, and at least one protein listed in Table I is provided. In one embodiment the complex comprises 1, 2, 3, or 4 of the proteins listed in Table I. Preferably the complex comprises at least the SNP containing TCF7L2 nucleic acid and PARP-1.

In another aspect of the invention, a method for identifying agents which disrupt the binding complexes described herein, thereby modulating TCF7L2 function is disclosed. An exemplary method comprises incubating said complex in the presence and absence of an effective amount of said agent, said complex comprising at least one detectably labeled protein or nucleic acid (step a); measuring disruption of said binding complex in the presence of said agent relative to that observed in the absence of said agent (step b), agents which disrupt said complex being identified as modulators TCF7L2 function. The method can be performed in vitro or in vivo within a cell. TCF7L2 functions to be modulated, include, without limitation, Wnt signaling, chromatin remodeling, activation of target gene expression and DNA damage detection and repair. Exemplary agents include, siRNAs, antisense oligonucleotides, small molecules, peptides and known inhibitors of PARP1 currently in clinical trials for the treatment of other disorders.

In yet another aspect of the invention, a method of increasing glucagon-like peptide 1 (GLP-1) secretion in a patient in need thereof is provided. An exemplary method entails administration of an effective amount of a PARP-1 inhibitor, the inhibitor being effective to increase GLP-1 secretion in a therapeutically beneficial manner. In a preferred embodiment, the patient has Type 2 diabetes and the increase in GLP-1 secretion alleviates diabetic symptoms in said patient. Exemplary PARP-1 inhibitors include for this purpose, include, without limitation, olaparib, rucaparib and iniparib.

### BRIEF DESCRIPTION OF THE DRAWINGS

FIG. 1 shows the results from oligo pull down for protein identification. A. biotin-labeled double-stranded oligonucleotides harboring SNP rs7903146 used for protein binding identification; B. Proteins from nuclear lysates of HCT116 cells (2 mg total protein each) that bind to biotin labeled, double-stranded oligonucleotides, stained with coomassie blue R-250. Protein bands uniquely in the rs7903146 oligo pull down as identified by the arrow.

FIG. 2A shows the flowchart of SILAC experiments. The HCT116 cells are metabolically labeled ¹³C6-lysine and ¹³C6-arginine to allow discrimination based on differences in peptide mass (4 Da). Biotinylated DNA oligo are synthesized and incubated with nuclear extracts from unlabeled (Lysine-d0) and labeled cells. Streptavidin beads are used to immobilize protein-DNA complex. After in-gel digestion with trypsin, differentially labeled forms of lysine and arginine containing tryptic peptides are detected and quantified by mass spectrometry (MS). FIG. 2B shows a table of quantitative alteration of Parp-1, RNA helicase A and Thrap3 binding after insulin treatment based on SILAC analysis. FIG. 2C shows relative binding amounts of Parp-1, RNA helicase A and Thrap3 determined using a bar chart format.

FIG. 3A shows the results obtained when HCT116 nuclear extracts were used for co-immunoprecipitation to demonstrate protein interaction. TCF7L2, Parp-1, RNA helicase A and Thrap3 interact with each other and form a complex. FIG. 3B is a schematic diagram illustrating complex formation between TCF7L2, Parp-1, RNA helicase and Thrap3 which appears to regulate chromatin remodeling, DNA repair, transcriptional regulation.

FIG. 4 shows the relative DNA binding affinity of XRCC5 and RP-A p70 between C and T allele of rs7903146. One peptide spectrum of XRCC5 is given.

FIG. 5 is a graph showing alterations in GLP-1 levels as determined by ELISA in basal and glucose treated NCI-H716 cells in the presence and absence of Olaparib, Rucaparib and Iniparib.

### DETAILED DESCRIPTION OF THE INVENTION

Resolving the underlying functional mechanism of a given genetic association has proven extremely challenging. However the key type 2 diabetes associated locus, TCF7L2, presents an opportunity for translational analyses, as studies in multiple ethnicities and with Baysian modeling strongly suggest that SNP, rs7903146, is the causal variant within this gene. This variant has also been associated with a variety of other disorders. We hypothesized that protein factors bind to this intronic region to modulate TCF7L2 function. We carried out oligo pull-down combined with mass spectrophotometry (MS) to elucidate the transcriptional machinery across the SNP. Nuclear lysates from HCT116 cells, where TCF7L2 is abundantly expressed, were incubated with biotin-labeled, double-stranded 60 bp oligonucleotides spanning rs7903146. The DNA-protein complexes were precipitated with streptavidin-agarose beads, and the bound proteins were isolated by denaturing SDS-PAGE. Following digestion with trypsin, the samples were analyzed by MS.

We observed that poly (ADP-ribose) polymerase 1 (PARP1) is by far the most abundant binding factor. We also used stable isotope labeling to quantify binding affinity changes following insulin treatment. Although PARP1 binding was modestly increased by 20%, among the next most abundant binding proteins, ATP-dependent RNA helicase A and thyroid hormone receptor-associated protein 3 (THRAP3) showed markedly increased binding of 12 and 7 fold, respectively. Interestingly, all three proteins dimerize with TCF7L2 itself, supporting the notion of an expression feedback loop. In addition, we found evidence for an allelic difference for proteins with less abundant binding, namely X-ray repair cross-complementing 5 (XRCC5) and RPNp70.

Our results point to a protein complex binding across rs7903146 within the TCF7L2 gene, that is both sensitive to insulin and allelic status, and reveal a mechanism by which this locus confers risk. Moreover, the protein DNA interactions described herein provide new druggable targets for use in screening assays to identify agents having efficacy for the treatment of metabolic disorders associated with the presence of this variant.

### DEFINITIONS

The following definitions are provided to facilitate an understanding of the present invention:

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. to encompass methods to prevent and/or to ameliorate the disease or disorder as well. The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim, an in the latter instance, impurities ordinarily associated with the specified material(s). The term “consisting essentially of” limits the scope of a claim to the specified elements, steps or materials and those that do not materially affect the basic and novel characteristic(s) of the claimed subject matter.

According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

“T2D-associated SNP or specific marker” is a SNP or marker which is associated with an increased or decreased risk of developing T2D not found normal patients who do not have this disease. Such markers may include but are not limited to nucleic acids, proteins encoded thereby, or other small molecules. Relevant information for the marker of the invention can be found in the dbSNP entry on the world wide web at .ncbi.nlm.nih.gov/SNP/snp_ref.cgi?type=rs&rs=rs7903146.

The phrase “Type 2 diabetes (T2D)” formerly noninsulin-dependent diabetes mellitus (NIDDM) or adult-onset diabetes) makes up about 90% of cases of diabetes. T2D is a metabolic disorder due to high blood glucose caused by insulin resistance and relative insulin deficiency. Without adequate insulin, glucose builds up in the bloodstream instead of going into the cells. The body is unable to use this glucose for energy despite high levels in the bloodstream, leading to increased hunger. In addition, the high levels of glucose in the blood causes the patient to urinate more, which in turn causes excessive thirst.

A “single nucleotide polymorphism (SNP)” refers to a change in which a single base in the DNA differs from the usual base at that position. These single base changes are called SNPs or “snips.” Millions of SNP's have been cataloged in the human genome. Some SNPs such that which causes sickle cell are responsible for disease. Other SNPs are normal variations in the genome.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

“Target nucleic acid” as used herein refers to a previously defined region of a nucleic acid present in a complex nucleic acid mixture wherein the defined wild-type region contains at least one known nucleotide variation which may or may not be associated with T1D. The nucleic acid molecule may be isolated from a natural source by cDNA cloning or subtractive hybridization or synthesized manually. The nucleic acid molecule may be synthesized manually by the triester synthetic method or by using an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence which hybridizes to the T2D specific marker gene or nucleic acid, but does not hybridize to other nucleotides. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the T1D specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the T1D specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably a T2D specific marker molecule. Samples may include but are not limited to cells, body fluids, including blood, serum, plasma, urine, saliva, tears, pleural fluid and the like.

### Methods of Using Protein DNA Complexes of the Invention to Screen Agents Useful for the Treatment of T2D

Since the protein DNA binding complexes identified herein have been associated with the etiology of T2D, methods for identifying agents that modulate the activity of the TCF7L2 SNP containing nucleic acids its interacting proteins should result in the generation of efficacious therapeutic agents for the treatment of a variety of disorders associated with this condition.

These DNA protein binding complexes provide suitable targets for the rational design of therapeutic agents which modulate the activity of the DNA binding proteins identified herein, thereby interfering with the T2D phenotype. Small nucleic acid molecules or peptides corresponding to these regions may be used to advantage in the design of therapeutic agents which effectively modulate the activity of the encoded proteins.

Molecular modeling should facilitate the identification of specific organic molecules with capacity to bind to the active site of the proteins which bind the SNP containing TCF7L2 nucleic acids based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Agents which can be tested for disruption of DNA/PARP-1 binding include, without limitation, those currently being tested in clinical trials for other disorders, such as cancer. Exemplary agents include, Iniparib (BSI 201), Olaparib (AZD-2281), Rucaparib (AG014699, PF-01367338), Veliparib (ABT-888), CEP 9722, MK 4827, Inhibitor of PARP1 and PARP2. BMN-673 and 3-aminobenzamide, a prototypical PARP inhibitor.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have the SNP containing TCF7L2 allele. The host cell lines or cells are grown in the presence of drug compound. The rate of cellular metabolism of the host cells is measured to determine if the compound is capable of regulating cellular metabolism or other cellular parameters associated with the diabetic phenotype. A variety of cell lines are commercially available for use in such screening assays. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

Cells and cell lines suitable for studying the effects of disrupting the interaction between the proteins and SNP containing TCF7L2 nucleic acids on glucose metabolism and methods of use thereof for drug discovery are provided. Such cells and cell lines will be transfected with the SNP encoding nucleic acids described herein and the effects on glucagon secretion, insulin secretion and/or beta cell apoptosis can be determined. Such cells and cell lines will also be contacted with the siRNA molecules provided herein to assess the effects thereof on glucagon secretion, insulin secretion and/or beta cell apoptosis. The siRNA molecules will be tested alone and in combination of 2, 3, 4, and 5 siRNAs to identify the most efficacious combinations. Cells suitable for these purposes include, without limitation, INS cells (ATCC CRL 11605), PC12 cells (ATCC CRL 1721), MIN6 cells, alpha-TC6 cells and INS-1 832/13 cells (Fernandez et al., J. of Proteome Res. (2007). 7:400-411). Pancreatic islet cells can be isolated and cultured as described in Joseph, J. et al., (J. Biol. Chem. (2004) 279:51049). Diao et al. (J. Biol. Chem. (2005) 280:33487-33496), provide methodology for assessing the effects of the SNP encoding nucleic acids and/or the siRNAs provided herein on glucagon secretion and insulin secretion. Park, J. et al. (J. of Bioch. and Mol. Biol. (2007) 40:1058-68) provide methodology for assessing the effect of these nucleic acid molecules on glucosamine induced beta cell apoptosis in pancreatic islet cells.

A wide variety of expression vectors are available that can be modified to express the sequences of this invention. The specific vectors exemplified herein are merely illustrative, and are not intended to limit the scope of the invention. Expression methods are described by Sambrook et al. Molecular Cloning: A Laboratory Manual or Current Protocols in Molecular Biology 16.3-17.44 (1989). Expression methods in Saccharomyces are also described in Current Protocols in Molecular Biology (1989).

Suitable vectors for use in practicing the invention include prokaryotic vectors such as the pNH vectors (Stratagene Inc., 11099 N. Torrey Pines Rd., La Jolla, Calif. 92037), pET vectors (Novogen Inc., 565 Science Dr., Madison, Wis. 53711) and the pGEX vectors (Pharmacia LKB Biotechnology Inc., Piscataway, N.J. 08854). Examples of eukaryotic vectors useful in practicing the present invention include the vectors pRc/CMV, pRc/RSV, and pREP (Invitrogen, 11588 Sorrento Valley Rd., San Diego, Calif. 92121); pcDNA3.1/V5&His (Invitrogen); baculovirus vectors such as pVL1392, pVL1393, or pAC360 (Invitrogen); and yeast vectors such as YRP17, YIPS, and YEP24 (New England Biolabs, Beverly, Mass.), as well as pRS403 and pRS413 Stratagene Inc.); Picchia vectors such as pHIL-D1 (Phillips Petroleum Co., Bartlesville, Okla. 74004); retroviral vectors such as PLNCX and pLPCX (Clontech); and adenoviral and adeno-associated viral vectors.

Promoters for use in expression vectors of this invention include promoters that are operable in prokaryotic or eukaryotic cells. Promoters that are operable in prokaryotic cells include lactose (lac) control elements, bacteriophage lambda (pL) control elements, arabinose control elements, tryptophan (trp) control elements, bacteriophage T7 control elements, and hybrids thereof. Promoters that are operable in eukaryotic cells include Epstein Barr virus promoters, adenovirus promoters, SV40 promoters, Rous Sarcoma Virus promoters, cytomegalovirus (CMV) promoters, baculovirus promoters such as AcMNPV polyhedrin promoter, Picchia promoters such as the alcohol oxidase promoter, and Saccharomyces promoters such as the gal4 inducible promoter and the PGK constitutive promoter, as well as neuronal-specific platelet-derived growth factor promoter (PDGF), and the Thy-1 promoter.

In addition, a vector of this invention may contain any one of a number of various markers facilitating the selection of a transformed host cell. Such markers include genes associated with temperature sensitivity, drug resistance, or enzymes associated with phenotypic characteristics of the host organisms.

Host cells expressing the T2D-associated SNP and the proteins that bind it or functional fragments thereof provide a system in which to screen potential compounds or agents for the ability to modulate the development of T2D. Thus, in one embodiment, the nucleic acid molecules of the invention may be used to create recombinant cell lines for use in assays to identify agents which modulate aspects of the diabetic phenotype. Also provided herein are methods to screen for compounds capable of modulating the function of proteins which bind the SNP containing nucleic acids described below.

Another approach entails the use of phage display libraries engineered to express fragment of the polypeptides bound by the SNP containing nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacophore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacophore.

Thus, one may design drugs which have, e.g., improved polypeptide activity or stability or which act as inhibitors, agonists, antagonists, etc. of polypeptide activity. By virtue of the availability of the binding complex between the proteins and SNP containing nucleic acid sequence described herein, sufficient amounts of the encoded complex may be made available to perform such analytical studies as x-ray crystallography. In addition, the knowledge of the protein sequence provided herein will guide those employing computer modeling techniques in place of, or in addition to x-ray crystallography.

### Pharmaceuticals and Peptide Therapies

The elucidation of the role played by the T2D associated SNP described herein in cellular metabolism facilitates the development of pharmaceutical compositions useful for treatment and diagnosis of T2D. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

As it is presently understood, RNA interference involves a multi-step process. Double stranded RNAs are cleaved by the endonuclease Dicer to generate nucleotide fragments (siRNA). The siRNA duplex is resolved into 2 single stranded RNAs, one strand being incorporated into a protein-containing complex where it functions as guide RNA to direct cleavage of the target RNA (Schwarz et al, Mol. Cell. 10:537 548 (2002), Zamore et al, Cell 101:25 33 (2000)), thus silencing a specific genetic message (see also Zeng et al, Proc. Natl. Acad. Sci. 100:9779 (2003)).

The invention includes a method of treating T2D in a mammal. An exemplary method entails administering to the mammal a pharmaceutically effective amount of an siRNA molecule directed to a PARP-1. Such molecules are commercially available from Dharmacon. The siRNA inhibits the expression of the aforementioned gene. Preferably, the mammal is a human. The term “patient” as used herein refers to a human.

Specific siRNA preparations directed at inhibiting the expression of PARP-1 as well as delivery methods are provided as a novel therapy to treat T2D. The siRNA can be delivered to a patient in vivo either systemically or locally with carriers, as discussed below. The compositions of the invention may be used alone or in combination with other agents or genes encoding proteins to augment the efficacy of the compositions.

A “membrane permeant peptide sequence” refers to a peptide sequence which is able to facilitate penetration and entry of the siRNA inhibitor across the cell membrane. Exemplary peptides include with out limitation, the signal sequence from Karposi fibroblast growth factor exemplified herein, the HIV tat peptide (Vives et al., J Biol. Chem., 272:16010-16017, 1997), Nontoxic membrane translocation peptide from protamine (Park et al., FASEB J. 19(11):1555-7, 2005), CHARIOT® delivery reagent (Active Motif; U.S. Pat. No. 6,841,535) and the antimicrobial peptide Buforin 2.

In one embodiment of the invention siRNAs are delivered for therapeutic benefit. There are several ways to administer the siRNA of the invention to in vivo to treat T2D including, but not limited to, naked siRNA delivery, siRNA conjugation and delivery, liposome carrier-mediated delivery, polymer carrier delivery, nanoparticle compositions, plasmid-based methods, and the use of viruses.

siRNA composition of the invention can comprise a delivery vehicle, including liposomes, for administration to a subject, carriers and diluents and their salts, and/or can be present in pharmaceutically acceptable formulations. This can be necessary to allow the siRNA to cross the cell membrane and escape degradation. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192; Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule.

The frequency of administration of the siRNA to a patient will also vary depending on several factors including, but not limited to, the type and severity of the T2D to be treated, the route of administration, the age and overall health of the individual, the nature of the siRNA, and the like. It is contemplated that the frequency of administration of the siRNA to the patient may vary from about once every few months to about once a month, to about once a week, to about once per day, to about several times daily.

Pharmaceutical compositions that are useful in the methods of the invention may be administered systemically in parenteral, oral solid and liquid formulations, ophthalmic, suppository, aerosol, topical or other similar formulations. In addition to the appropriate siRNA, these pharmaceutical compositions may contain pharmaceutically-acceptable carriers and other ingredients known to enhance and facilitate drug administration. Thus such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to administer the appropriate siRNA to a patient according to the methods of the invention. The use of nanoparticles to deliver siRNAs, as well as cell membrane permeable peptide carriers that can be used are described in Crombez et al., Biochemical Society Transactions v35:p 44 (2007).

Methods of the invention directed to treating T2D involve the administration of at least one PARP-1 directed siRNA in a pharmaceutical composition. The siRNA is administered to an individual as a pharmaceutical composition comprising the siRNA and a pharmaceutically acceptable carrier. Pharmaceutically acceptable carriers are well known in the art and include aqueous solutions such as physiologically buffered saline, other solvents or vehicles such as glycols, glycerol, oils such as olive oil or injectable organic esters.

A pharmaceutically acceptable carrier can contain physiologically acceptable compounds that act, for example, to stabilize the siRNA or increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the siRNA.

One skilled in the art appreciates that a pharmaceutical composition comprising siRNA can be administered to a subject by various routes including, for example, orally or parenterally, such as intravenously (i.v.), intramuscularly, subcutaneously, intraorbitally, intranasally, intracapsularly, intraperitoneally (i.p.), intracisternally, intra-tracheally (i.t.), or intra-articularly or by passive or facilitated absorption. The same routes of administration can be used other pharmaceutically useful compounds, for example, small molecules, nucleic acid molecules, peptides, antibodies and polypeptides as discussed hereinabove.

A pharmaceutical composition comprising siRNA inhibitor also can be incorporated, if desired, into liposomes, microspheres, microbubbles, or other polymer matrices (Gregoriadis, Liposome Technology, Vols. I to III, 2nd ed., CRC Press, Boca Raton Fla. (1993)). Liposomes, for example, which consist of phospholipids or other lipids, are nontoxic, physiologically acceptable and metabolizable carriers that are relatively simple to make and administer.

The pharmaceutical preparation comprises a siRNA targeting the SNP containing sequences described herein or an expression vector encoding for the siRNA. Such pharmaceutical preparations can be administered to a patient for treating T2D.

Expression vectors for the expression of siRNA molecules preferably employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502 09).

A formulated siRNA composition can be a composition comprising one or more siRNA molecules or a vector encoding one or more siRNA molecules independently or in combination with a cationic lipid, a neutral lipid, and/or a polyethyleneglycol-diacylglycerol (PEG-DAG) or PEG-cholesterol (PEG-Chol) conjugate. Non-limiting examples of expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500-505.

A lipid nanoparticle composition is a composition comprising one or more biologically active molecules independently or in combination with a cationic lipid, a neutral lipid, and/or a polyethyleneglycol-diacylglycerol (i.e., polyethyleneglycol diacylglycerol (PEG-DAG), PEG-cholesterol, or PEG-DMB) conjugate. In one embodiment, the biologically active molecule is encapsulated in the lipid nanoparticle as a result of the process of providing and aqueous solution comprising a biologically active molecule of the invention (i.e., siRNA), providing an organic solution comprising lipid nanoparticle, mixing the two solutions, incubating the solutions, dilution, ultrafiltration, resulting in concentrations suitable to produce nanoparticle compositions.

Nucleic acid molecules can be administered to cells by incorporation into other vehicles, such as biodegradable polymers, hydrogels, cyclodextrins. (see for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. US 2002130430), biodegradable nanocapsules, and bioadhesive microspheres, or by proteinaceous vectors (O'Hare and Normand, International PCT Publication No. WO 00/53722)

Cationic lipids and polymers are two classes of non-viral siRNA delivery which can form complexes with negatively charged siRNA. The self-assembly PEG-ylated polycation polyethylenimine (PEI) has also been used to condense and protect siRNAs (Schiffelers et al., 2004, Nuc. Acids Res. 32: 141-110). The siRNA complex can be condensed into a nanoparticle to allow efficient uptake of the siRNA through endocytosis. Also, the nucleic acid-condensing property of protamine has been combined with specific antibodies to deliver siRNAs and can be used in the invention (Song et al., 2005, Nat Biotech. 23:709-717).

In order to treat an individual having T2D, to alleviate a sign or symptom of the disease, siRNA should be administered in an effective dose. The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. One skilled in the art would know that the amount of siRNA required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having T2D.

The effective dose of siRNA will depend on the mode of administration, and the weight of the individual being treated. The dosages described herein are generally those for an average adult but can be adjusted for the treatment of children. The dose will generally range from about 0.001 mg to about 1000 mg.

The concentration of siRNA in a particular formulation will depend on the mode and frequency of administration. A given daily dosage can be administered in a single dose or in multiple doses so long as the siRNA concentration in the formulation results in the desired daily dosage. One skilled in the art can adjust the amount of siRNA in the formulation to allow administration of a single dose or in multiple doses that provide the desired concentration of siRNA over a given period of time.

In an individual suffering from T2D, in particular a more severe form of the disease, administration of siRNA can be particularly useful when administered in combination, for example, with a conventional agent for treating such a disease. The skilled artisan would administer siRNA, alone or in combination and would monitor the effectiveness of such treatment using routine methods such as pancreatic beta cell function determination, radiologic, immunologic or, where indicated, histopathologic methods. Other conventional agents for the treatment of diabetes include insulin administration, glucagon administration or agents that alter levels of either of these two molecules. Glucophage®, Avandia®, Actos®, Januvia® and Glucovance® are examples of such agents.

Administration of the pharmaceutical preparation is preferably in an “effective amount” this being sufficient to show benefit to the individual. This amount prevents, alleviates, abates, or otherwise reduces the severity of T2D symptoms in a patient.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

### Kits and Articles of Manufacture

Any of the aforementioned products can be incorporated into a kit which may contain an T2D-associated SNP specific marker TCF7L2 polynucleotide or one or more such markers immobilized on a Gene Chip, an oligonucleotide, a polypeptide which binds the SNP containing nucleic acid described herein, a peptide designed to disrupt such binding, an siRNA, a small molecule, an antibody, a label, marker, or reporter, a pharmaceutically acceptable carrier, a physiologically acceptable carrier, instructions for use, a container, a vessel for administration, an assay substrate, or any combination thereof.

The methods set forth below are provided to facilitate the practice of the present invention.

### Cell Culture and Nuclear Extracts Preparation

Human HCT 116 cells were cultured in DMEM (4.5 g/l glucose, 10% FCS, 100 U/mL penicillin and 100 íg/mL streptomycin). For insulin stimulation experiments, cells were treated with 1 uM insulin for 1 hour. For SILAC experiments, Cultures were grown according to standard cell-culture procedures in SILAC-light and SILAC-heavy labeled Dulbecco's Modified Eagle Medium (DMEM, ThermoFisher) supplied with 10% dialyzed fetal bovine serum (FBS), ¹³C6-lysine and ¹³C6-arginine. Cells were washed by cold phosphate-buffered saline (PBS) and harvested by scraping, centrifugation for 5 min at 5000 rpm at 4° C. 10⁸ Cells were lysed in 250 ul low salt buffer (10 mM HEPES, 1.5 mM MgCl2, 10 mM KCl, 0.5% NP-40, proteasome inhibitor, phosphatase inhibitor, 10% glycerol, pH 7.9) by incubation on ice for 20 min with vortex 10 seconds per 5 minutes. The nuclei were separated from cytoplasmic fraction by centrifugation 10,000 rpm for 1 min at 4° C. The nuclei pellets were washed once by cold low salt buffer, harvested by centrifugation. The nuclei proteins were released by high salt buffer (20 mM HEPES, 1.5 mM MgCl2, 420 mM NaCl, 0.2 mM EDTA, proteasome inhibitor, phosphatase inhibitor, 25% glycerol, pH 7.9) and supernatant were collected by centrifugation 12,000 rpm for 10 min at 4° C.

### Oligonucleotide Pull Down

The SNP rs7903146 5′ Dual Biotin modified oligonucleotides were synthesized by Integrated DNA Technologies, Inc. The sequences of oligonucleotides were as follows:

### C Allele:

For:
5′-ACAATTAGAGAGCTAAGCACTTTTTAGATACTATATAATTTAATTG
CCGTATGAGGCACCC-3′
Rev:
5′-GGGTGCCTCATACGGCAATTAAATTATATAGTATCTAAAAAGTGCT
TAGCTCTCTAATTGT-3′

### T Allele:

For:
5′-ACAATTAGAGAGCTAAGCACTTTTTAGATATTATATAATTTAATTG
CCGTATGAGGCACCC-3′
Rev:
5′-GGGTGCCTCATACGGCAATTAAATTATATATATCTAAAAAGTGCTT
AGCTCTCTAATTGT-3′

10 μm forward and reverse oligonucleotides were annealed prior to use. 4 picomole of oligonucleotide was mixed with 1 mg of nuclear extract in binding buffer (20 mM HEPES, 1.5 mM MgCl2, 150 mM NaCl, 0.2 mM EDTA, proteasome inhibitor, phosphatase inhibitor, 25% glycerol, pH 7.9) in a total volume of 1 ml. Mixtures were incubated for 1 hr at 4° C. on a rotator. 60 microliters of streptavidin was added to each tube, and the mixture was further incubated for 30 min 4° C. on a rotator. Beads were spun for 30 s at 800 g and then washed 5×500 μl in binding buffer. The supernatant was discarded, and proteins were eluted by boiling in SDS sample buffer. The eluates were isolated by SDS-page gel following by Coomassie blue R-250 staining. The interested bands were excised from the gel and digested by trypsin for mass spectrometry.

### Mass Spec

The sample was digested with trypsin and analyzed with nanoLC/MS/MS at the Penn Proteomics Core. University of Pennsylvania, supported by grant P30CA016520 (Abramson Cancer Center), and by grant ES013508-04 (CEET). The data were analyzed with Sequest (ThermoFinnigan, San Jose, Calif.; version SRF v. 5). Scaffold (version Scaffold 3.6.0, Proteome Software Inc., Portland, Oreg.) was used to validate MS/MS based peptide and protein identifications. For SILAC experiment, Zoom Scan is added to mass spec method to monitor the labeled and unlabeled peptides. The quantification of the SILAC peaks was performed with ProteolQ with customized modification so that the software can use the zoom scanned SLICA peaks for quantification.

### Immunoprecipitation and Western Blotting

Co-immunoprecipitation was performed using IP buffer (50 mM tris, 150 mM NaCl, proteasome inhibitor, phosphatase inhibitor, 10% glycerol, pH 8.0) on a rotator at 4° C. for overnight. Western blotting was performed according to standard procedures. The following antibodies were used: anti-TCF7L2 (Millipore, 05-511), anti-Parp-1 (cell signaling, 46D11), anti-RNA helicase A (Abcam, Ab26271) and anti-THRAP3 (Novus, NB100-40848).

The following examples are provided to illustrate embodiments of the invention. They are not intended to limit the invention in any way.

### Example 1

### Parp-1, RNA Helicase A and Thrap3 Interact with the DNA Elements Surrounding the SNP rs7903146 within Intron 3 of the TCF7L2 Gene

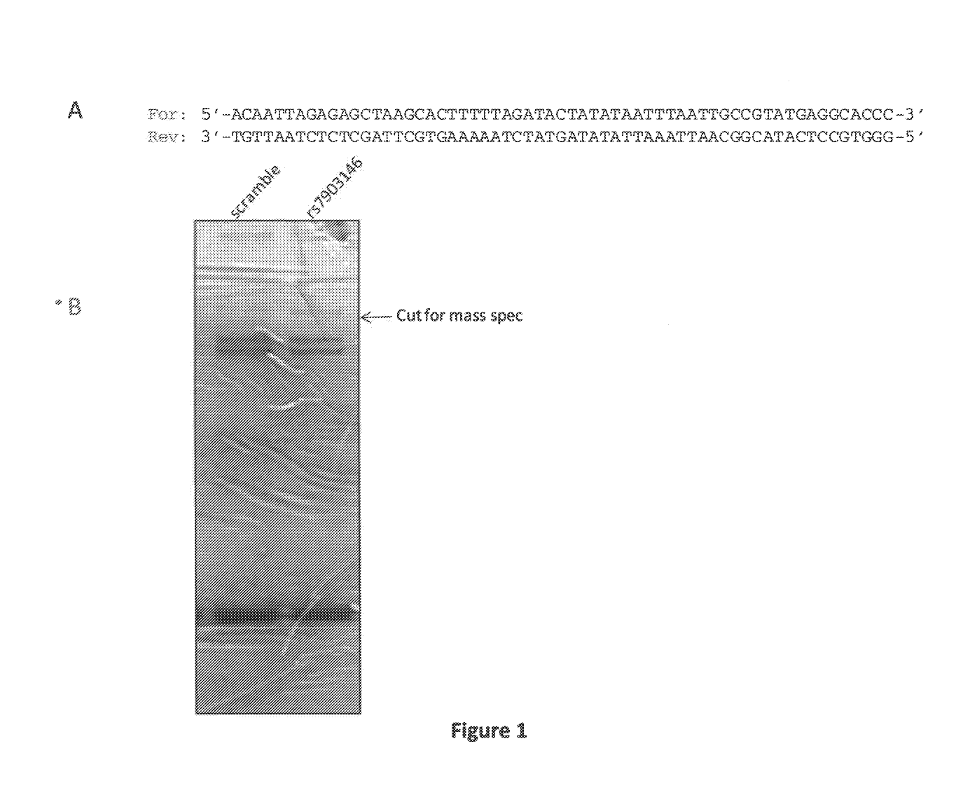

To elucidate the role of the SNP rs7903146 for TCF7L2 T2D associated activity, we hypothesized that trans-acting protein factors bind to this genomic region to modulate the TCF7L2 function. To test our hypothesis, we used mass spectrometer to identify several proteins interacting with the element containing the SNP rs7903146. See Table I. Nuclear lysates from HCT116 cells were incubated with biotin-labeled, double-stranded oligonucleotide spanning the SNP rs7903146 (FIG. 1A). The DNA-protein complexes were precipitated with streptavidin-agarose beads, and the bound proteins were isolated by denaturing SDS-PAGE followed by staining with coomassie blue R-250. As shown in FIG. 1B, several bands were visible in the pull-down samples. One major band was seen in the SNP rs7903146 samples but not in the scramble samples. This specific band was cut from the gel, digested with trypsin and followed by LC-MS/MS analysis. We set a cut-off to N=10 for the number of identified peptide. The identified proteins above the cut-off are listed in table 1, ordered by peptide abundance from top to bottom. Here we focused on three abundant proteins: Poly [ADP-ribose] polymerase 1 (Parp-1), which was dominantly binding plus ATP-dependent RNA helicase A and Thyroid hormone receptor-associated protein 3 (Thrap3), which were insulin responsive—see ‘Sample4 C PARP BAND 110 kD ins’).

TABLE I
Identity of the proteins in bands Src Ia-c, with the top protein in each box
being the most abundantly identity by MS.

| | Total |
|---|---|
| Name | Spectra |
| Poly [ADP-ribose] polymerase 1 | 108 |
| cDNA FLJ53442, highly similar to Poly (ADP-ribose) polymerase 1 | 106 |
| DNA topoisomerase 1 | 42 |
| ATP-dependent RNA helicase A | 39 |
| Isoform Beta of DNA ligase 3 | 29 |
| Isoform Alpha of DNA ligase 3 | 29 |
| FACT complex subunit SPT16 | 19 |
| Thyroid hormone receptor-associated protein 3 | 18 |
| Isoform 1 of Nucleolar RNA helicase 2 | 18 |
| Isoform 2 of Nucleolar RNA helicase 2 | 18 |
| Nucleolin | 17 |
| cDNA FLJ45706 fis, clone FEBRA2028457, highly similar to | 16 |
| Nucleolin | |
| DNA damage-binding protein 1 | 16 |
| Isoform 1 of Splicing factor 3B subunit 3 | 13 |
| Isoform 1 of Myb-binding protein 1A | 12 |
| Isoform 2 of Myb-binding protein 1A | 12 |
| NCL protein | 12 |
| X-ray repair cross-complementing protein 5 | 12 |
| Putative uncharacterized protein NOP2 | 11 |
| NOP2 protein | 11 |
| Isoform 2 of Putative ribosomal RNA methyltransferase NOP2 | 11 |
| 32 kDa protein | 11 |
| cDNA FLJ51067, highly similar to DNA damage-binding protein 1 | 11 |
| 116 kDa U5 small nuclear ribonucleoprotein component | 10 |
| 116 kDa U5 small nuclear ribonucleoprotein component isoform b | 10 |
| Myosin-Id | 10 |
| Isoform 1 of Putative ATP-dependent RNA helicase DHX30 | 10 |
| Isoform 3 of Putative ATP-dependent RNA helicase DHX30 | 10 |
| Isoform 2 of Putative ATP-dependent RNA helicase DHX30 | 10 |
| Putative uncharacterized protein DHX30 | 10 |
| Isoform 1 of Aspartyl/asparaginyl beta-hydroxylase | 10 |
| aspartyl/asparaginyl beta-hydroxylase isoform f | 10 |

To confirm our findings, we then performed Western blotting for the oligo pull-down samples. As illustrated in FIG. 1C, Parp-1, ATP-dependent RNA helicase A and Thrap3 demonstrated the association with the oligonucleotide containing the SNP rs7903146. This experiment provides additional in vitro evidence suggesting that these three proteins interact with this genomic region.

It was well established that Parp-1 plays a role in DNA damage detection and repair (Waldman and Waldman 1991; Schultz, Lopez et al. 2003; Godon, Cordelieres et al. 2008). Recent studies have revealed important roles for PARP-1 in chromatin and transcriptional regulation (Kraus and Lis 2003; Tulin, Chinenov et al. 2003; Kim, Zhang et al. 2005; Ju, Lunyak et al. 2006; Krishnakumar, Gamble et al. 2008; Doege, Inoue et al. 2012). RNA helicases can unwind double-stranded DNA and RNA in a 3′ to 5′ direction and thereby regulate the interactions between proteins and DNA or RNA (Weidensdorfer, Stohr et al. 2009). RNA helicases participate in multiple biological processes including transcription, splicing and translation (Schmid and Linder 1992; Lauber, Fabrizio et al. 1996; Luking, Stahl et al. 1998; Tetsuka, Uranishi et al. 2004; Abdelhaleem 2005; Cordin, Banroques et al. 2006). Thrap3 functions as the activator of pre-mRNA splicing and immediate mRNA degradation (Lee, Hsu Ia et al. 2010). Recent research demonstrated that Thrap3 is also associated with DNA damage (Beli, Lukashchuk et al. 2012). However, the underlying mechanisms of Thrap3 remain poorly understood. Our data suggest an important role of Thrap3 for the transcription of TCF7L2.

### The Binding Affinity of Parp-1, ATP-Dependent RNA Helicase A and Thrap3 Increased Following Insulin Stimulation

To investigate dynamic binding of Parp-1, ATP-dependent RNA helicase A and Thrap3 for the SNP rs7903146 region, we used stable isotope labeling with amino acids in cell culture (SILAC) (Ong, Blagoev et al. 2002) to quantify the binding affinity changes between with and without insulin treatment.

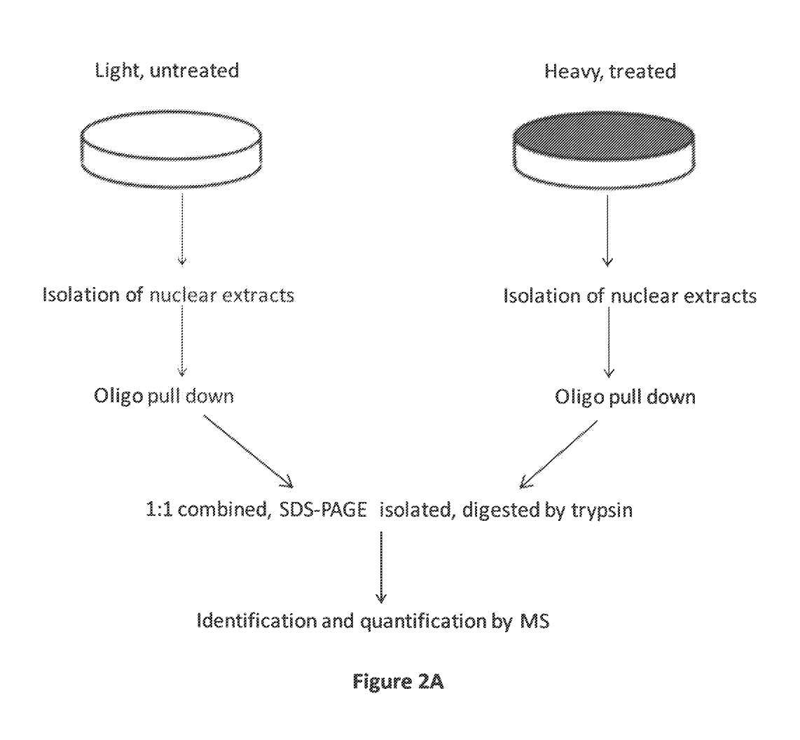

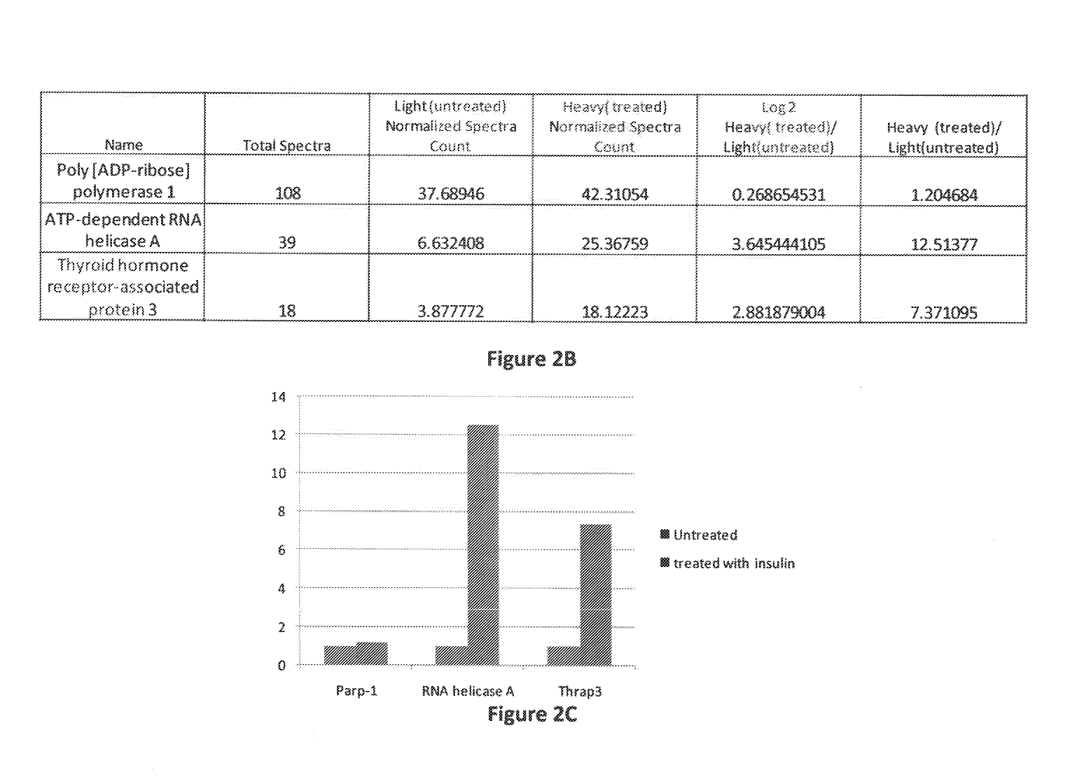

The heavy cells were isotopically labeled in SILAC medium supplied with ¹³C6-lysine and ¹³C6-arginine and treated with insulin, while the light cells isotopically labeled served as a control. Equal amounts of nuclear extracts from the two SILAC-labeled cell populations were used for oligo pull down. After pull down, the light and heavy were mixed, isolated with SDS page gel, digested by trypsin and followed by mass spec analysis. With this approach, we quantified the relative protein abundances including Parp-1, ATP-dependent RNA helicase A and Thrap3 between treatment and without treatment of insulin. As shown FIG. 2, parp-1 was slightly up-regulated by 20% upon stimulation and ATP-dependent RNA helicase A and Thrap3 significantly increased up to 12 and 7 fold, respectively. Collectively, our results demonstrated that TCF7L2 appears to be regulated by ATP-dependent RNA helicase A and Thrap3 via insulin stimulation.

### TCF7L2, Parp-1, RNA Helicase A and Thrap3 Form a Complex in HCT116

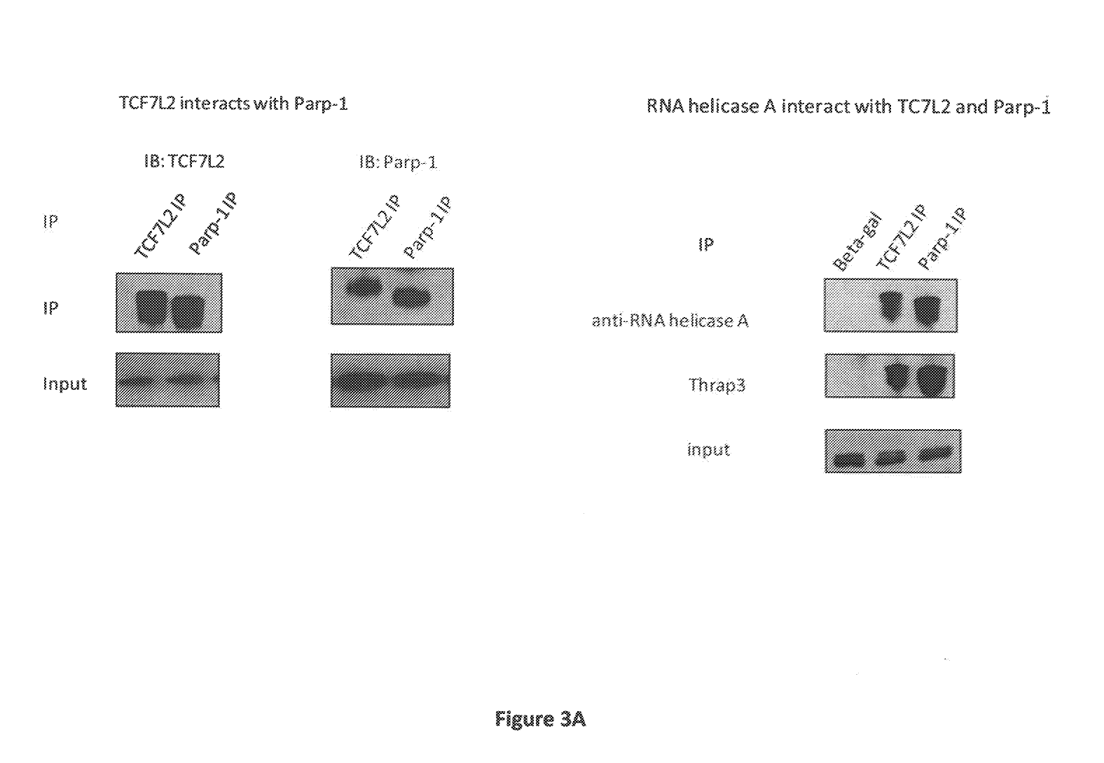

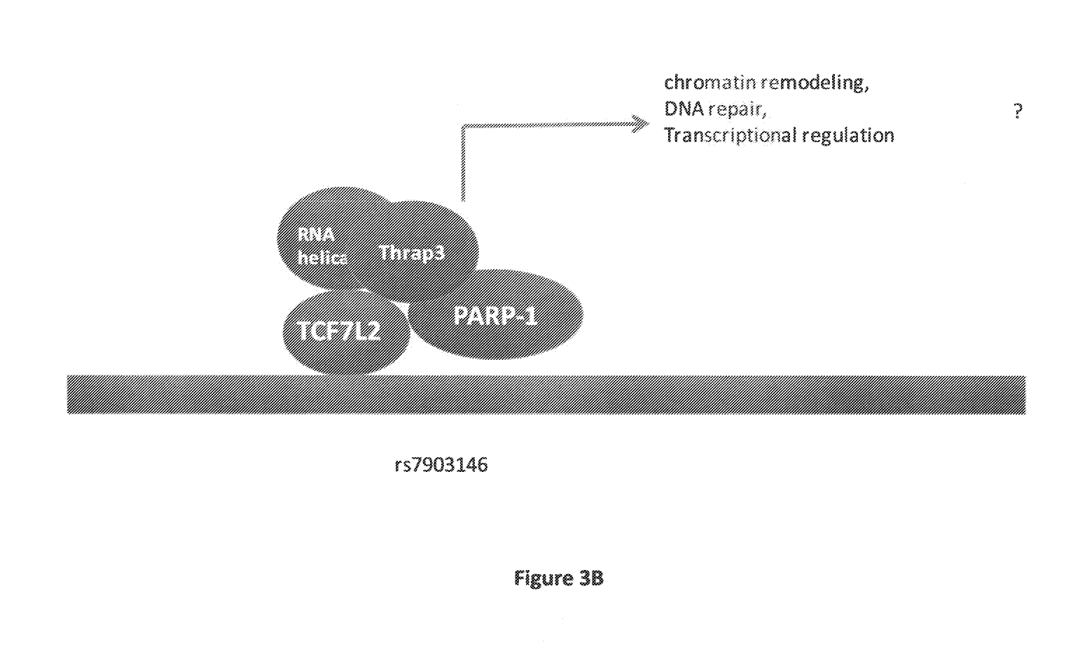

Since Parp-1, RNA helicase A and Thrap3 were identified from the same oligo pull down samples, we tried to characterize the interactions among them to understand how these factors are involved in TCF7L2 genomic organization. We propose that they may interact with each other to form a complex bound to SNP rs7903146 region. In order to test this hypothesis, we performed co-immunoprecipitation experiments. As shown in FIG. 3A, parp-1 exhibited the strong interaction with RNA helicase A and Thrap3. Interestingly, we also found that TCF7L2 interacts with these proteins. Our previous TCF7L2 chip-seq results have characterized four sites within TCF7L2 genome (Zhao, Schug et al. 2010). Collectively, we propose a possible mechanism that TCF7L2 may interact with Parp-1, RNA helicase A and Thrap3 to form a complex to regulate TCF7L2 itself (FIG. 3B). More detailed studies of these proteins are being performed to further characterize the functional effects of these interactions.

X-Ray Repair Cross-Complementing Protein 5 and Replication Protein A 70 kDa DNA-Binding Subunit and Preferentially Bind to T Allele of rs7903146

Several independent studies have demonstrated that the T-allele of the transcription factor 7-like 2 gene (TCF7L2) polymorphism rs7903146 is associated with type 2 diabetes (T2D)(Grant, Thorleifsson et al. 2006; Saxena, Gianniny et al. 2006; Scott, Bonnycastle et al. 2006; Helgason, Palsson et al. 2007; Palmer, Hester et al. 2011). The work with multiple ethnicities has identified the association to the single variant rs7903146 in intron 3. Thereby, SNP rs7903146 is considered as the causal diabetes susceptibility variant by the whole community. However, the mechanism by which the SNP exerts its effect on TCF7L2 function is unknown. We propose there are allelic-preferential binding proteins across the SNP and therefore regulate TCF7L2 function. To test our hypothesis, we performed “two-way” oligo pull down experiments. The nuclear extracts from the light cells were isotopically labeled in SILAC medium was used pull down experiments by C allele oligo while the nuclear extracts from the heavy cells were isotopically labeled in SILAC medium was used pull down experiments by T allele oligo in forward experiments. The SILAC labeled extracts were switched in reverse experiments. This design can improve the reliability and reproducibility of the approach.

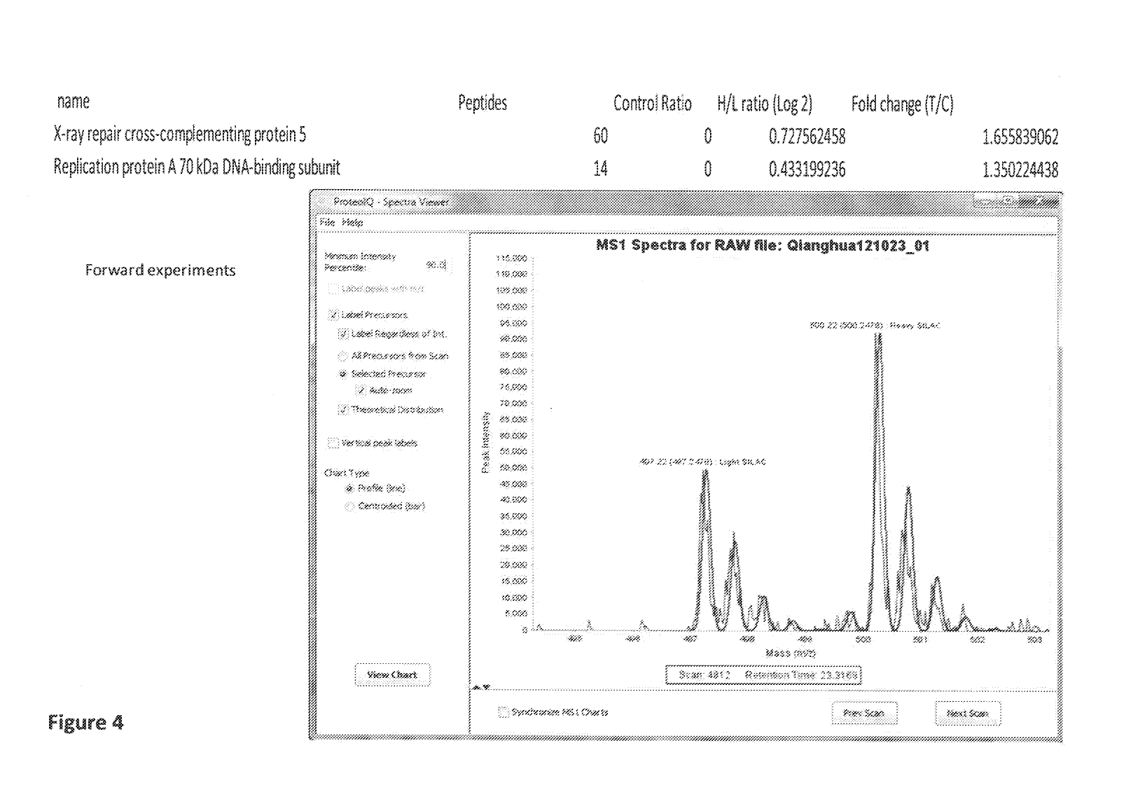

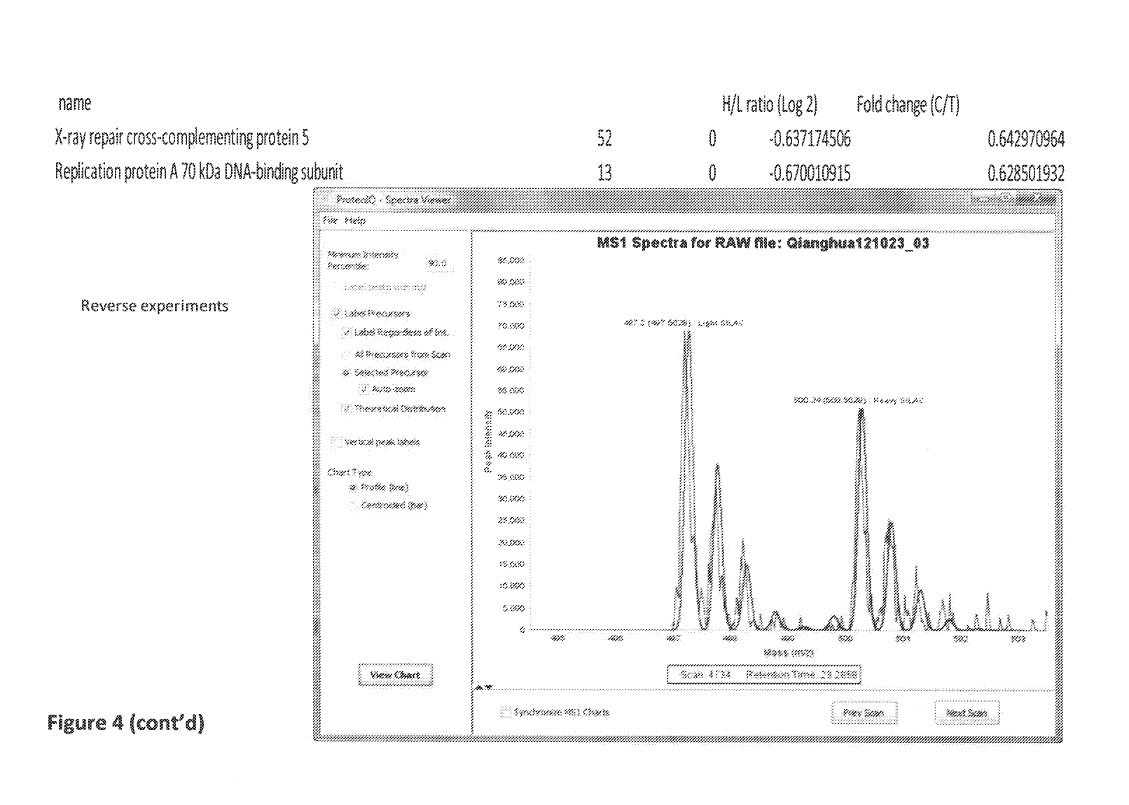

Interestingly, we identified two proteins X-ray repair cross-complementing protein 5 (XRCC5) and replication protein A 70 kDa DNA-binding subunit preferentially bind to T allele over C allele (FIG. 4). The consistent results from two-way pull down suggest that these two proteins may play a role in the regulation of TCF7L2 by an allele specific binding preference. XRCC5 is a component of ATP-dependent DNA helicase II complex. The XRCC5/6 heterodimer is involved in DNA damage and repair (Taccioli, Gottlieb et al. 1994; Roberts, Strande et al. 2010). It was also reported that the XRCC5/6 heterodimer acts as a negative regulator of the transcriptional activity of the ETS Factor ESE-1 through interaction with p300, CREB-binding protein (Wang, Fang et al. 2004). These results suggest that XRCC5/6 may regulate TCF7L2 though a DNA binding affinity mechanism.

The replication protein A 70 kDa DNA-binding subunit (RP-A p70), also called replication factor A protein 1, belongs to the replication factor A protein 1 family composed of three subunit proteins RPA1, RPA2 and RPA3 (Umbricht, Griffin et al. 1994). It participates in multiple biological processes in DNA metabolism, including recombination, replication, damage and repair (Mason, Haring et al. 2009; Kemp, Mason et al. 2010). RP-A p70 interacts with the DNA polymerase catalytic subunit POLA1/p180 and control the fidelity of DNA replication (Braun, Lao et al. 1997; Ikegami, Kuraoka et al. 1998). RP-A p70 plays an important role in cell cycle progression. Several studies have reported that abolishment of RPA1 by RNAi results in G2/M arrest in cell cycle and subsequent increases cell death (Dodson, Shi et al. 2004; Haring, Mason et al. 2008). It was a well established notion that the limited capacity of adult β-cells to proliferate and beta-cell loss caused by increased beta-cell apoptosis is an important mechanism contributing to the onset of type 2 diabetes (Rhodes 2005). However, it is not yet clear whether regulation of RP-A p70 activity contributes to the β-cell dysfunction of type 2 diabetes.

It should be noted that the difference in DNA binding affinity of these two proteins between C and T allele are moderate. In fact, the odds ratio for the (T) risk allele of rs7903146 is 1.45 in GWAS studies. Odds ratios reported in GWA studies are usually small because common diseases are caused by a large number of causal variants with small effect sizes (Stringer, Wray et al. 2011). Thereby, the development of new approaches for reliable and quantitative analysis in biological processes is necessary. Quantitative and high through proteomics like mass spec therefore provide powerful tools for post-GWAS functional studies.

As described herein above, the proteins listed in Table I provide novel targets for the development of new therapeutic agents efficacious for the treatment of T2D. In particular, it would be desirable to block expression of these genes in those patients that are more prone to develop the disease. In this regard, the therapeutic siRNAs described herein can be used to block expression of the gene product based on the patient signal, thereby inhibiting the pancreatic β-cell destruction that occurs in T2D.

### REFERENCES

Abdelhaleem, M. (2005). “RNA helicases: regulators of differentiation.” Clin Biochem 38(6): 499-503.
Beli, P., N. Lukashchuk, et al. (2012). “Proteomic investigations reveal a role for RNA processing factor THRAP3 in the DNA damage response.” Mol Cell 46(2): 212-225.
Braun, K. A., Y. Lao, et al. (1997). “Role of protein-protein interactions in the function of replication protein A (RPA): RPA modulates the activity of DNA polymerase alpha by multiple mechanisms.” Biochemistry 36(28): 8443-8454.
Cordin, O., J. Banroques, et al. (2006). “The DEAD-box protein family of RNA helicases.” Gene 367: 17-37.
Dodson, G. E., Y. Shi, et al. (2004). “DNA replication defects, spontaneous DNA damage, and ATM-dependent checkpoint activation in replication protein A-deficient cells.” J Biol Chem 279(32): 34010-34014.
Doege, C. A., K. Inoue, et al. (2012). “Early-stage epigenetic modification during somatic cell reprogramming by Parp1 and Tet2.” Nature 488(7413): 652-655.
Godon, C., F. P. Cordelieres, et al. (2008). “PARP inhibition versus PARP-1 silencing: different outcomes in terms of single-strand break repair and radiation susceptibility.” Nucleic Acids Res 36(13): 4454-4464.
Grant, S. F., G. Thorleifsson, et al. (2006). “Variant of transcription factor 7-like 2 (TCF7L2) gene confers risk of type 2 diabetes.” Nat Genet 38(3): 320-323.
Grove, E. A. (2011). “Wnt signaling meets internal dissent.” Genes Dev 25(17): 1759-1762.
Haring, S. J., A. C. Mason, et al. (2008). “Cellular functions of human RPA1. Multiple roles of domains in replication, repair, and checkpoints.” J Biol Chem 283(27): 19095-19111.
Helgason, A., S. Palsson, et al. (2007). “Refining the impact of TCF7L2 gene variants on type 2 diabetes and adaptive evolution.” Nat Genet 39(2): 218-225.
Ikegami, T., I. Kuraoka, et al. (1998). “Solution structure of the DNA- and RPA-binding domain of the human repair factor XPA.” Nat Struct Biol 5(8): 701-706.
Ju, B. G., V. V. Lunyak, et al. (2006). “A topoisomerase IIbeta-mediated dsDNA break required for regulated transcription.” Science 312(5781): 1798-1802.
Kemp, M. G., A. C. Mason, et al. (2010). “An alternative form of replication protein a expressed in normal human tissues supports DNA repair.” J Biol Chem 285(7): 4788-4797.
Kim, M. Y., T. Zhang, et al. (2005). “Poly(ADP-ribosyl)ation by PARP-1: ‘PAR-laying’ NAD+ into a nuclear signal.” Genes Dev 19(17): 1951-1967.
Kraus, W. L. and J. T. Lis (2003). “PARP goes transcription.” Cell 113(6): 677-683.
Krishnakumar, R., M. J. Gamble, et al. (2008). “Reciprocal binding of PARP-1 and histone H1 at promoters specifies transcriptional outcomes.” Science 319(5864): 819-821.
Kumar, V. F., Nelson; Abbas, Abul K.; Cotran, Ramzi S.; Robbins, Stanley L. (2005). “Robbins and Cotran Pathologic Basis of Disease (7th ed.).”: 1194-1195.
Lauber, J., P. Fabrizio, et al. (1996). “The HeLa 200 kDa U5 snRNP-specific protein and its homologue in Saccharomyces cerevisiae are members of the DEXH-box protein family of putative RNA helicases.” EMBO J 15(15): 4001-4015.
Lee, K. M., W. Hsu Ia, et al. (2010). “TRAP150 activates pre-mRNA splicing and promotes nuclear mRNA degradation.” Nucleic Acids Res 38(10): 3340-3350.
Logan, C. Y. and R. Nusse (2004). “The Wnt signaling pathway in development and disease.” Annu Rev Cell Dev Biol 20: 781-810.
Luking, A., U. Stahl, et al. (1998). “The protein family of RNA helicases.” Crit Rev Biochem Mol Biol 33(4): 259-296.
Mason, A. C., S. J. Haring, et al. (2009). “An alternative form of replication protein a prevents viral replication in vitro.” J Biol Chem 284(8): 5324-5331.
Ong, S. E., B. Blagoev, et al. (2002). “Stable isotope labeling by amino acids in cell culture, SILAC, as a simple and accurate approach to expression proteomics.” Mol Cell Proteomics 1(5): 376-386.
Palmer, N. D., J. M. Hester, et al. (2011). “Resequencing and analysis of variation in the TCF7L2 gene in African Americans suggests that SNP rs7903146 is the causal diabetes susceptibility variant.” Diabetes 60(2): 662-668.
Ravindranath, A., A. O'Connell, et al. (2008). “The role of LEF/TCF factors in neoplastic transformation.” Curr Mol Med 8(1): 38-50.
Rhodes, C. J. (2005). “Type 2 diabetes—a matter of beta-cell life and death?” Science 307(5708): 380-384.
Roberts, S. A., N. Strande, et al. (2010). “Ku is a 5′-dRP/AP lyase that excises nucleotide damage near broken ends.” Nature 464(7292): 1214-1217.
Saxena, R., L. Gianniny, et al. (2006). “Common single nucleotide polymorphisms in TCF7L2 are reproducibly associated with type 2 diabetes and reduce the insulin response to glucose in nondiabetic individuals.” Diabetes 55(10): 2890-2895.
Schmid, S. R. and P. Linder (1992). “D-E-A-D protein family of putative RNA helicases.” Mol Microbiol 6(3): 283-291.
Schultz, N., E. Lopez, et al. (2003). “Poly(ADP-ribose) polymerase (PARP-1) has a controlling role in homologous recombination.” Nucleic Acids Res 31(17): 4959-4964.
Scott, L. J., L. L. Bonnycastle, et al. (2006). “Association of transcription factor 7-like 2 (TCF7L2) variants with type 2 diabetes in a Finnish sample.” Diabetes 55(9): 2649-2653.
Stringer, S., N. R. Wray, et al. (2011). “Underestimated effect sizes in GWAS: fundamental limitations of single SNP analysis for dichotomous phenotypes.” PLoS One 6(11): e27964.
Taccioli, G. E., T. M. Gottlieb, et al. (1994). “Ku80: product of the XRCC5 gene and its role in DNA repair and V(D)J recombination.” Science 265(5177): 1442-1445.
Tetsuka, T., H. Uranishi, et al. (2004). “RNA helicase A interacts with nuclear factor kappaB p65 and functions as a transcriptional coactivator.” Eur J Biochem 271(18): 3741-3751.
Tulin, A., Y. Chinenov, et al. (2003). “Regulation of chromatin structure and gene activity by poly(ADP-ribose) polymerases.” Curr Top Dev Biol 56: 55-83.
Umbricht, C. B., C. A. Griffin, et al. (1994). “High-resolution genomic mapping of the three human replication protein A genes (RPA1, RPA2, and RPA3).” Genomics 20(2): 249-257.
van Es, J. H., P. Jay, et al. (2005). “Wnt signalling induces maturation of Paneth cells in intestinal crypts.” Nat Cell Biol 7(4): 381-386.
Waldman, A. S. and B. C. Waldman (1991). “Stimulation of intrachromosomal homologous recombination in mammalian cells by an inhibitor of poly(ADP-ribosylation).” Nucleic Acids Res 19(21): 5943-5947.
Wang, H., R. Fang, et al. (2004). “Positive and negative modulation of the transcriptional activity of the ETS factor ESE-1 through interaction with p300, CREB-binding protein, and Ku 70/86.” J Biol Chem 279(24): 25241-25250.
Weidensdorfer, D., N. Stohr, et al. (2009). “Control of c-myc mRNA stability by IGF2BP1-associated cytoplasmic RNPs.” RNA 15(1): 104-115.
Zhao, J., J. Schug, et al. (2010). “Disease-associated loci are significantly over-represented among genes bound by transcription factor 7-like 2 (TCF7L2) in vivo.” Diabetologia 53(11): 2340-2346.

### Example II

The TCF7L2 variant has been also been associated with other sub-forms of diabetes. These include for example, cystic fibrosis related diabetes (Blackman S M, et al. Diabetologia. 2009 September; 52(9):1858-65; latent autoimmune diabetes in adults (Cervin et al. Diabetes 2008 May; 57(5):1433-7; Lukacs et al. Diabetologia. 2012 March; 55(3):689-93), gestational diabetes (Mayo H, et al. PLoS One. 2012; 7(9):e45882; J Matern Fetal Neonatal Med. 2012 September; 25(9):1783-6) and islet anti-antibody-negative diabetes in young patients (Yu et al., J Clin Endocrinol Metab. 2009 February; 94(2):504-10.

The presence of the TCF7L2 variant also influences normal human development. For example, Freathy et al. have shown that TCF7L2 risk genotypes alter birth weight in a study of 24,053 individuals Am J Hum Genet. 2007 June; 80(6):1150-61). The variant has also been associated with premature adrenarche (early puberty). See for example, Lappalainen S, et al. Metabolism. 2009 September; 58(9):1263-9).

As mentioned above, the variant is associated with several different metabolic disorders which may or may not be associated with diabetic complications. For example, Yan et al. have shown that the transcription factor 7-like 2 (TCF7L2) polymorphism may be associated with focal arteriolar narrowing in Caucasians with hypertension or without diabetes (BMC Endocr Disord. 2010 May 17; 10:9; also see Melzer et al. BMC Med. 2006 Dec. 20; 4:34.

TCF7L2 gene polymorphisms have also been associated with diabetic retinopathy and cardiovascular autonomic neuropathy (Ciccacci C, et al. Acta Diabetol. 2012 Jul. 28; Luo et al. Diabetes. 2013 Feb. 22.

Cardiovascular complications have also been observed in patients with this variant. The TCF7L2 polymorphism rs7903146 has been associated with coronary artery disease severity and mortality. See Sousa A G, et al. PLoS One. 2009 Nov. 17; 4(11):e7697. The variant also has effects on lipid metabolism of three different populations as described by Perez-Martinez P, et al PLoS One. 2012; 7(8):e43390.

Several researchers report that single nucleotide polymorphisms of TCF7L2 are linked to diabetic coronary atherosclerosis. See Muendlein A, et al. PLoS One. 2011 Mar. 15; 6(3):e17978 and Kucharska-Newton A M, et al. J Obes. 2010; 2010.

This TCF7L2 variant has also been associated with neuropsychiatric disorders, such as schizophrenia in an Arab-Israeli family sample. (Alkelai A, et al. PLoS One. 2012; 7(1):e29228. Hansen et al. report an increased risk of schizophrenia in patients harboring the variant. Biol Psychiatry. 2011 Jul. 1; 70(1):59-63. Also see Irvin et al, (Schizophr Res. 2009 October; 114(1-3):50-6) who describe genetic risk factors for type 2 diabetes with pharmacologic intervention in African-American patients with schizophrenia or schizoaffective disorder.

Associations between TCF7L2 polymorphisms and cancer have been described in the literature. Increased risk of breast cancer among Hispanic and non-Hispanic white women: the Breast Cancer Health Disparities Study, have been described by Connor A E, et al. (Breast Cancer Res Treat. 2012 November; 136(2):593-602). The single nucleotide polymorphisms appear to impact wnt signaling pathway genes with breast cancer in saudi patients as reported by Alanazi M S, et al. PLoS One. 2013; 8(3):e59555. Also see Naidu et al., Med Oncol. 2012 June; 29(2):411-7. The variant also appears to modulate colorectal cancer risk. See Sainz J, et al. J Clin Endocrinol Metab. 2012 May; 97(5):E845-51. Variation in TCF7L2 and increased risk of colon cancer: the Atherosclerosis Risk in Communities (ARIC) Study was described by Folsom A R, al. Diabetes Care. 2008 May; 31(5):905-9).

Genetic variants in TCF7L2 and KCNJ11 genes in a Greek population have been associated with polycystic ovary syndrome. (Christopoulos P. et al., Gynecol Endocrinol. 2008 September; 24(9):486-90. The variant has also been associated with CKD progression and renal function in population-based cohorts. See Kottgen A, et al., (J Am Soc Nephrol. 2008 October; 19(10):1989-99).

In view of the association of the TCF7L2 variant with each of the disorders listed above, it is clear that agents which alter the activity of the variant may have significant clinical benefit. Accordingly agents which disrupt protein complex formation at this variant are highly desirable and readily identifiable using the screening methods provided hereinabove.

Another important use of this variant entails a test and treat paradigm of disease management. Patients will be tested for the presence or the absence of the variant, if the variant is present this will guide the clinician as to the proper use of currently available and newly developed therapeutic agents. The identification of the various components present in the TCF7L2 transcriptional complex provides new avenues for the development such therapeutic agents.

### Example 3

### Parp-1 Inhibition Promotes Glucagon-Like Peptide-1 (GLP-1) Secretion

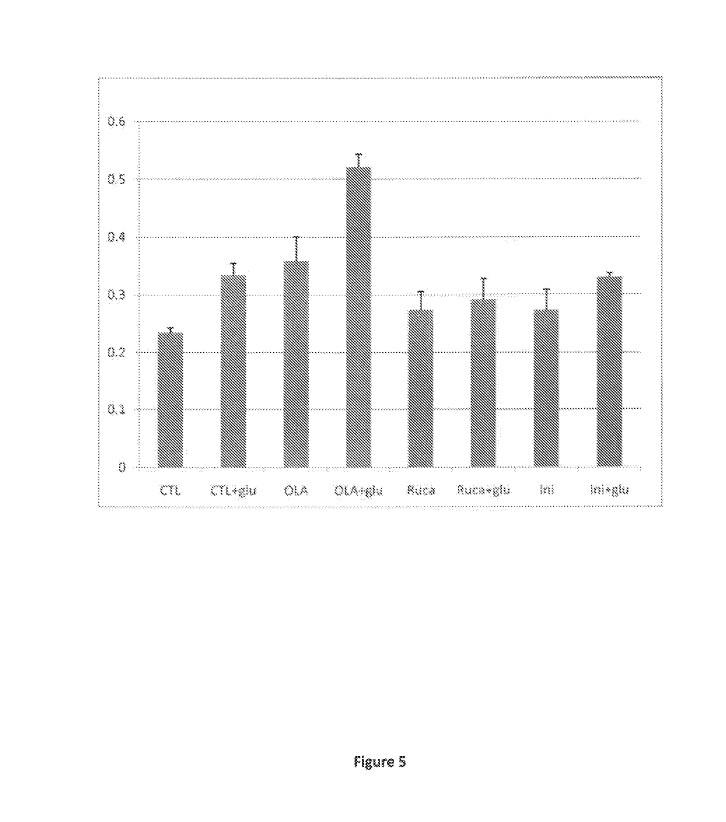

Glucagon-like peptide-1 (GLP-1) has numerous physiological functions, including enhancement of glucose-stimulated insulin secretion, stimulation of β-cell anti-apoptosis and proliferation and inhibition of glucagon secretion, food intake and gastric emptying. These antidiabetic properties of GLP-1 have generated intense interest in the use of this short peptide and its agonists for the treatment of patients with type 2 diabetes. A better understanding of the underlying mechanism of GLP-1 secretion may lead to novel approaches used for the treatment of type 2 diabetes. TCF7L2 is the most strongly associated locus with type 2 diabetes and has been known to bind with the promoter region of the proglucagon gene for many years. In addition, using oligo-pull down followed by mass-spec, we identified in the previous examples the specific protein factors binding across the presumed causal variant, rs7903146 within TCF7L2, the most abundant of which was PARP-1; interestingly, there is already some literature suggesting that TCF7L2 and PARP-1 work together plus PARP-1 knockout mice are protected from induced diabetes. As such, we investigated if existing PARP-1 inhibitors, previously tested in oncology studies, could have a GLP-1 agonist effect. We leveraged the standard human L-cell line, NCI-H716, for GLP-1 secretion assessment to investigate this possible link. We pre-treated the cells for 48 hours with three different PARP-1 inhibitors, Olaparib, Rucaparib and Iniparib, with or without glucose stimulation (16.8 mM). We measured the resulting GLP-1 concentration using an ELISA kit (Millipore). The data show that basal levels of GLP-1 were elevated by glucose. However, in the absence of glucose but in the presence of inhibitor, an equal or greater level of GLP-1 secretion was observed. Moreover, this increase in secretion was elevated further by the addition of glucose. See FIG. 5. In view of this result, it appears that Parp-1 inhibitors increase GLP-1 secretion and thus present a novel therapeutic strategy for the treatment of type 2 diabetes.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. An isolated binding complex comprising a SNP containing transcription factor 7-like 2 (TCF7L2) encoding nucleic acid, wherein said SNP is rs7903146, and at least one protein listed in Table I.

2. The isolated binding complex of claim 1, wherein said complex comprises 1, 2, 3, or 4 of the proteins listed in Table I.

3. The isolated binding complex of claim 2, wherein said 1 protein is PARP-1.

4. The isolated binding complex of claim 2, wherein said 1 protein is Thrap3.

5. The isolated binding complex of claim 2, wherein said 1 protein is RNA helicase A.

6. A method for identifying agents which disrupt the binding complex of claim 1, thereby modulating TCF7L2 function, comprising;
a) incubating said complex in the presence and absence of an effective amount of said agent, said complex comprising at least one detectably labeled protein or nucleic acid;
b) measuring disruption of said binding complex in the presence of said agent relative to that observed in the absence of said agent, agents which disrupt said complex being identified as modulators of TCF7L2 function.

7. The method of claim 6, wherein said method is performed in a cell and said TCF7L2 function is selected from the group consisting of Wnt signaling, chromatin remodeling, activation of target gene expression and DNA damage detection and repair.

8. The method of claim 7, wherein said agent is selected from the group consisting of a siRNA, an antisense oligonucleotide, a small molecule, and a peptide.

9. The method of claim 7 wherein said cells are selected from the group consisting of INS cells, PC12 cells, MIN6 cells, pancreatic beta islet cells and alpha TC6 cells.

10. The method of claim 8 wherein modulatory effects of said siRNAs on a parameter selected from the group consisting of insulin secretion, glucagon secretion and glucosamine induced beta cell apoptosis is determined.

11. A method for enhancing glucagon-like peptide-1 (GLP-1) secretion in a patient in need thereof, comprising contacting GLP-1 producing cells with an effective amount of a PARP-1 inhibitor, said inhibitor stimulating secretion of GLP-1 and beta cell anti-apotosis and inhibiting glucagon secretion, thereby treating symptoms associated with diabetes.

12. The method of claim 11, further comprising administration of glucose to said patient.

13. The method of claim 11, wherein said diabetes is selected from the group consisting of type 2 diabetes, cystic fibrosis related diabetes, latent autoimmune diabetes, gestational diabetes, and islet anti-antibody-negative diabetes.

14. The method of claim 13, further comprising administration of an agent conventionally used to treat diabetes selected from the group consisting of insulin, glucagon, Glucophage®, Avanida®, Actos®, Januvia® and Glucovance®.

15. The method of claim 11, wherein said PARP-1 inhibitor is selected from the group consisting of Iniparib, Olaparib, Rucaparib, Veliparib, CEP 9722, MK 4827, BMN-673 and 3 aminobenzamide.


SEQUENCE LISTING (4 sequences)

SEQ ID NO: 1 - DNA, 61 bp, Artificial Sequence
  Primer
acaattagagagctaagcactttttagatactatataatttaattgccgtatgaggcaccc

SEQ ID NO: 2 - DNA, 61 bp, Artificial Sequence
  Primer
gggtgcctcatacggcaattaaattatatagtatctaaaaagtgcttagctctctaattgt

SEQ ID NO: 3 - DNA, 61 bp, Artificial Sequence
  Primer
acaattagagagctaagcactttttagatattatataatttaattgccgtatgaggcaccc

SEQ ID NO: 4 - DNA, 61 bp, Artificial Sequence
  Primer
gggtgcctcatacggcaattaaattatataatatctaaaaagtgcttagctctctaattgt